(19) United States
(12) Patent Application Publication
(10) Pub. No.: US 2007/0028923 A1
(43) Pub. Date: Feb. 8, 2007

(54) Tip-laryngeal airway

(75) Inventors: Nicholas Souris, Smyrna, GA (US); Patrick Souris, Smyrna, GA (US)

Correspondence Address: NICHOLAS SOURIS, 331 HURT ROAD SW, SMYRNA, GA, 30082, US

(73) Assignee: Patrick Souris

(21) Appl. No.: 11/198,574

(22) Filed: Aug. 8, 2005

(51) Int. Cl. A61M 16/00

(52) U.S. Cl. 128/207.15

(57) ABSTRACT

An airway device used to establish an airway in an unconscious person or person receiving general anesthesia. The device is composed of a gas tube with a breathing circuit connector on one end and an endplate on the other. The present art incorporates a guide cord, guide loops or conduits, a pull-ring, endplate creases, tubular notches and geometric gas tube designs. Specifically, the guide cord is attached to the endplate tip and routed through the endplate and gas tube by means of the guide loops or conduits. The guide cord exits near the gas tube connector and terminates to a pull-ring. The guide cord conveys pull-ring manipulation to the endplate and permits change of endplate attitude and shape and prevents endplate fold back. Also, endplate creases, gas tube notches and geometric gas tube designs are applied to optimize insertion and safety of the device.

### CROSS-REFERENCE TO RELATED APPLICATIONS

Kamen U.S. Pat. No. 3,640,282
Kamen U.S. Pat. No. 3,799,173
Brain U.S. Pat. No. 4,509,514
Brain U.S. Pat. No. 5,282,464
Brain U.S. Pat. No. 5,632,271
Chang APP 20040200479
Chang WL 2320806
Kamen U.S. Pat. No. 5,988,167
Cook U.S. Pat. No. 5,937,860 B1
Cook U.S. Pat. No. 6,705,321 B2

### STATEMENT REGARDING FEDERALLY SPONSORED RESEARCH OR DEVELOPMENT

Not Applicable.

### INCORPORATION-BY-REFERENCE OF MATERIAL SUBMITTED ON A COMPACT DISC

Not Applicable.

### BACKGROUND OF INVENTION

1. Field of the Invention

This invention relates to contemporary laryngeal mask airway devices or supraglottic airway devices. Particularly, this is a device that seats or seals over the laryngeal opening to allow control of the airway in an unconscious person or a person under general anesthesia.

The present art airway device consists of a gas tube of suitable length with a breathing circuit connector on one end and an endplate of suitable thickness on the other end. The posterior to anterior boundaries of the endplate have a pharyngeal smooth side and a laryngeal face side. The laryngeal face side has the opening of the gas tube to allow medical gas to pass through the tube and out from the laryngeal face side to the person's laryngeal opening. Hence, this side of the endplate is a conduit from the gas tube to the person's laryngeal opening. To better seal or seat the laryngeal face side over the person's laryngeal opening, the overall shape of the endplate, in the laryngeal to pharyngeal view, is oval-teardrop shape with the smaller curve of the oval-teardrop shape distal to the adjoining gas tube. The gas tube is adjoined in the mid-line but off center at the portion of the endplate that has the larger curve of the oval-teardrop shape. The gas tube adjoins on the pharyngeal side of the endplate and makes approximately a 10 to 35 degrees angle at the joining point. Furthermore, the outer rim of the endplate is an inflatable and or pliable cuff that is able to conform over the person's laryngeal opening. This enhances the sealing or seating of the endplate over the laryngeal opening. The cuff is inflatable by means of tubing connected to a pilot balloon having a universal female locking check valve connector.

The insertion of the contemporary laryngeal mask airway device requires the endplate to enter the oral cavity and then ultimately have the endplate seat over the person's laryngeal opening with a portion of the gas tube extending out of the mouth and the gas tube connector connected to a breathing circuit.

The normal insertion of the contemporary laryngeal mask airway device into the person's oral pharynx is often difficult. One frequent reason is the endplate tip of the contemporary airway device tends to fold back upon itself in the pharynx causing a mal-positioning at the person's laryngeal opening. Hence the airway control is not established when the laryngeal mask airway is mal-positioned. Said mal-position of the endplate may cause trauma to the surrounding oral tissue.

Furthermore, additional oral trauma may be caused by multiple repositioning and insertion of the laryngeal mask airway device when attempting to establish a person's airway. Often an operator has to place his or her finger(s) or an instrument such as a tongue blade in the oral cavity to guide the airway device through the person's pharynx. This process of digital or instrument manipulation could cause additional trauma to the person's oral cavity. Furthermore, these manipulations increase the risk of cross contamination to the person.

In addition, establishing an airway with the laryngeal mask airway device is a time critical procedure. Delay in correct placement of the airway device can lead to person hypoxia. Hypoxia is a condition under which the person lacks adequate oxygen in the body.

The present art introduces an airway device with components to avoid said malfunction or mal-positioning. These components are a guide cord, guide loops or conduits, a pull-ring, endplate creases, tubular notches and geometric gas tube designs. These components will encourage optimal shape of the endplate and gas tube for insertion and placement of the airway device.

The guide cord is attached to the endplate tip and routed through the endplate and gas tube by means of the guide loops or conduits. Furthermore, the guide cord is exited near the gas tube connector and terminates to a pull-ring. The guide cord conveys pull-ring manipulation to the endplate and permits control of the endplate attitude. The desired endplate attitude is straight and or slightly elevated. Maintaining endplate attitude prevents the endplate from folding back upon itself herein referred to as endplate malfunction. Once the endplate is guided through the oral pharynx and placed over the laryngeal opening the operator completes insertion and placement of the airway device by releasing the pull-ring to relax the endplate.

The ability to effect endplate attitude and shape allows the operator dynamic control of the endplate during insertion. This dynamic control allows the operator to manipulate the endplate through various resistances in the oral cavity and therefore, permits a preferred guidance during insertion. Hence, adjusting the endplate by pulling the guide cord to prevent endplate mal-positioning will increase success of correct airway device placement.

2. Description of Related Art

There are two basic types of airway devices used in the unconscious person that this invention will discuss as prior art. These devices are the endotracheal tube, reference Kamen U.S. Pat. Nos. 3,640,282 and 3,799,173 and the laryngeal mask airway, reference Brain U.S. Pat. Nos. 4,509,514, 5,282,464 and 5,632,271 or similar. These airway devices both provide an airway passage for the person but are very different in functional design.

The endotracheal tube is intended to go through the person laryngeal opening that is through the vocal cords. Hence the endotracheal tube establishes an airway by having the tip of the tube in the person's tracheal and the other end connected to a breathing circuit. Whereas, the laryngeal airway device is intended to seat in front of the person's laryngeal opening that is seat over the vocal cords.

The laryngeal airway device is also known has a supraglottic airway device that is an airway device that does not go through the vocal cords. Hence the laryngeal airway establishes an airway by having the endplate lie over the laryngeal opening and the other end connected to the breathing circuit.

Consequently, the important point is that the laryngeal airway device does not go through the laryngeal opening that is not through the vocal cords, but lies over the laryngeal opening. Hence both devices establish an airway but have a significant difference in where the tube tip lies with respect to the person's vocal cords. Furthermore, with respect to the tube tip both devices have specific problems that affect successful placement.

A frequent problem with the contemporary laryngeal mask airway device is during insertion the endplate tends to fold back upon itself in the oral pharynx and does not maintain a desired position. Hence, this folding of the endplate causes difficulty in inserting the airway device through the oral pharynx. This results in a mal-position and ultimately a malfunction of the airway device. There have been attempts to resolve the problem of endplate malfunction.

Frequently, the operator either places his or her finger(s), or an instrument such as a tongue blade, into the oral cavity to help guide the laryngeal mask airway's endplate through the oral pharynx. This manipulation by the operator can result in trauma or cross contamination to the person. Furthermore, this manipulation causes the operator to use both hands when inserting the laryngeal mask airway device.

The ideal laryngeal airway device will maintain shape integrity during insertion, prevent endplate fold back, be constructed of soft or pliable materials, have the capacity to conform to various person anatomies and permit operator directional control during insertion.

Prior art attempts to prevent endplate fold back include a dual air chamber endplate, reference Chang patent # APP 20040200479 and WL 2320806, and a less flexible endplate construction. These designs increase the size and or the rigidity of the endplate. An increased size and or relatively harder or rigid material translate more pressure to the person's surrounding soft tissue. Hence these designs may promote irritation and increase the risk of sore throat to the person.

Another way to avoid the endplate fold back is to construct the endplate of an inflexible or semi-rigid material, reference Kamen U.S. Pat. No. 5,988,167. This attempt to overcome endplate fold back can result in a noticeable disadvantage to the person.

For instance, the more inflexible the material is, the more rigid the material is, albeit plastic, rubber or other. A relatively harder material passed over or laid against the person oral tissue may cause soft tissue irritation. Moreover an endplate that has rolls, “ribs” or rows of increase material thickness, reference Cook U.S. Pat. No. 5,937,860 B1 and U.S. Pat. No. 6,705,321 B2, in an attempt to prevent endplate fold back may promote soft tissue irritation. As a result, a sore throat is not an uncommon complaint from a person having received a laryngeal airway device constructed with a relatively firm or inflexible endplate.

Therefore, an ideal laryngeal airway device should have a soft endplate. That is an endplate with a soft material and a cuff that is high volume and low pressure. This will translate less pressure to the person's surrounding soft tissue. However, the softer the endplate the more risk the endplate may fold back upon itself during insertion. This is where the present art will enhance the ideal airway device endplate by reducing the possibility of endplate malfunction. Principally, in the present art applying tension to the guide cord maintains endplate shape integrity and avoids endplate fold back while maintaining the advantage of a soft and pliable endplate.

Other problems that the prior arts have not successfully resolved are the capacity to conform to various person anatomies and permit operator directional control during airway device insertion.

The oral pharynx is a curved passageway varying in size and resistance from the mouth opening to the laryngeal opening. Moreover, the oral pharynx varies in size according to individual person anatomy. In this consideration dynamic control over the endplate during insertion is important.

The present art allows the operator to respond to resistance sensed during insertion by adjusting endplate attitude and width. In the present art the operator feels the resistance on the endplate during insertion and can pull the guide cord more or less via pull-ring manipulation and ease the endplate through the oral pharynx to reside over the person's laryngeal opening.

Specifically, modulation of guide cord tension during insertion, achieved by pull-ring manipulation, permits the operator to ease the endplate through the oral pharynx to rest over the person's laryngeal opening. Whereas the prior art mentioned, such as a dual air chamber endplate or more rigid endplate inventions, offers only static control of the endplate and therefore the operator is less able to manipulate the endplate during insertion.

### BRIEF SUMMARY OF THE INVENTION

The present art is a laryngeal airway device used for an unconscious person needing an airway established or a person receiving general anesthesia. The primary object of this invention is to improve the airway device insertion through the oral pharynx and placement over the laryngeal opening.

The insertion of the contemporary laryngeal mask airway device into the oral pharynx is often difficult. One reason is the endplate of the contemporary device tends to fold back upon itself and thus cause resistance to further passage in the oral pharynx. This will result in a mal-positioning of the endplate over the laryngeal opening.

The present art introduces a laryngeal airway device with components added to avoid endplate malfunction and airway device mal-positioning. These components include a guide cord that is attached to the endplate tip and routed through the device and exited out near the gas tube connector. The guide cord then terminates to a pull-ring or other similar finger controller.

There are also strategically located guide loops or conduits that attached the guide cord onto the device. Having the guide cord attached at certain locations on the endplate and gas tube with the loops or conduits will allow specific tension to be applied onto the device when the guide cord is pulled. Pulling of the guide cord will therefore cause attitude and shape change of the airway device. Hence a desirable attitude and shape of the endplate and gas tube occurs when tension is applied to the guide cord.

Most importantly is attitude and shape control of the endplate during insertion. Consequently, the operator can apply tension to the endplate tip via pull-ring manipulation that conveys guide cord tension onto the endplate. This applied tension will maintain the endplate straight and or slightly elevated and to not fold back upon it's self. Once the airway device is placed in the oral pharynx and the endplate over the laryngeal opening the operator releases the guide cord and, thus accomplishes a successful device insertion and placement.

In addition there are strategically placed endplate creases, gas tube notches and geometric gas tube designs that contribute to an easier endplate insertion and successful device placement.

Also the present art airway device allows for single-handed insertion. Single-handed insertion avoids instrumentation and or digital manipulation that are often needed during contemporary device insertion. Furthermore, avoidance of instruments or operator's fingers into the oral cavity reduces risk of trauma and cross contamination to the person. In addition, a higher rate of successful placement adds to the reliability and efficiency of establishing an airway for the person. Foremost, this is a time critical process and by reducing the time to establish the airway device reduces the risk of hypoxia to the person.

### THE DRAWINGS

The present art, as to the arrangement and mode of operation, can be more fully understood from the following expositions when it is considered in conjunction with the accompanying drawings.

These figures partially show the object in accordance with the invention in a schematic fashion and are not to be taken to scale. The object of the individual figures is partially reduced or enlarged so that the construction can be more easily illustrated.

### BRIEF DESCRIPTION OF THE DRAWINGS

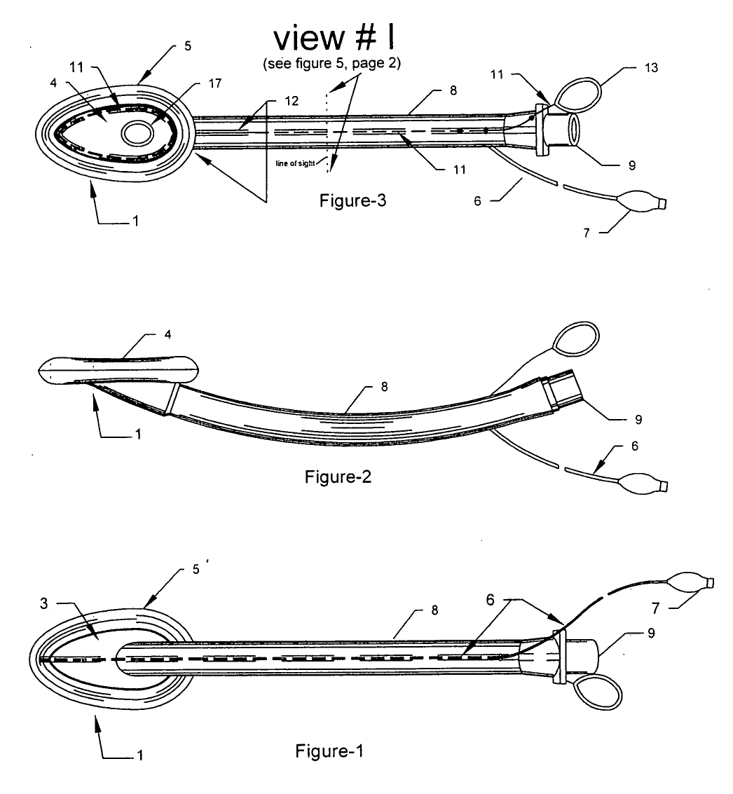

FIG. 1 shows a perspective posterior view of the airway device.

FIG. 2 shows a perspective lateral view of the airway device.

FIG. 3 shows a perspective anterior view of the airway device.

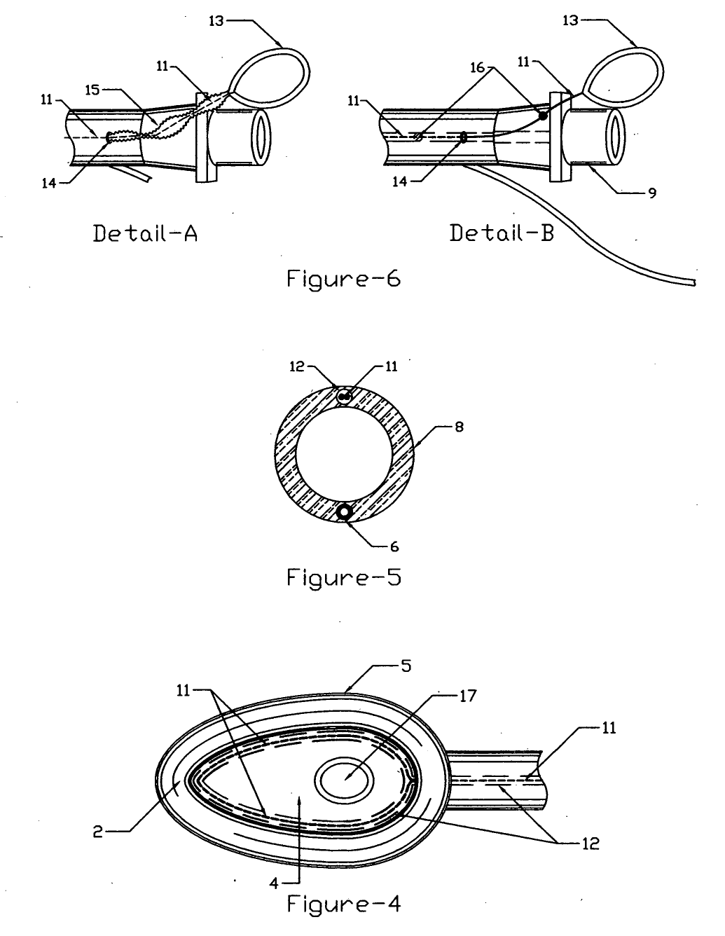

FIG. 4 shows a detail perspective view of the laryngeal face of the endplate.

FIG. 5 shows a detail perspective cross sectional view of the gas tube.

FIG. 6 shows two detail perspective anterior views, A & B, of the gas tube connector.

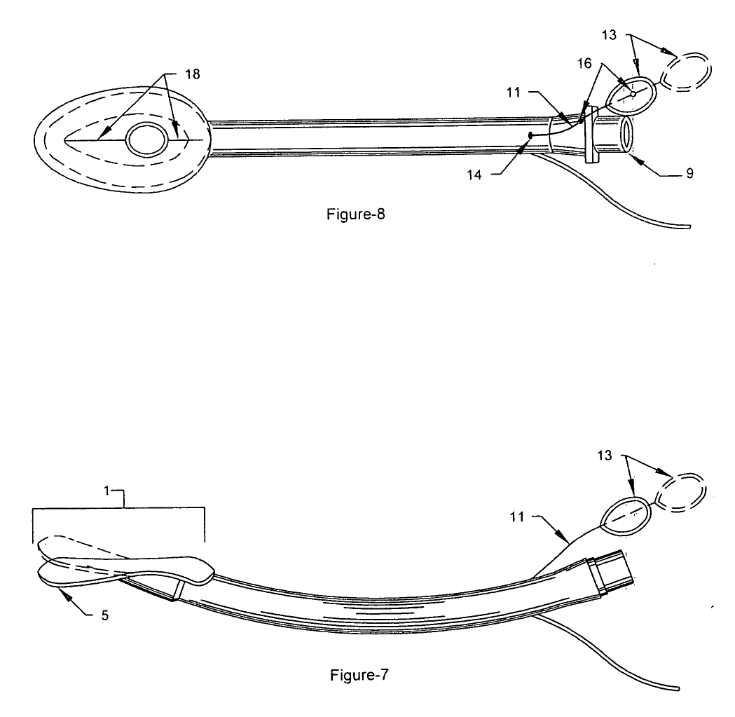

FIG. 7 shows an operational lateral view of the airway device.

FIG. 8 shows an operational anterior view of the airway device.

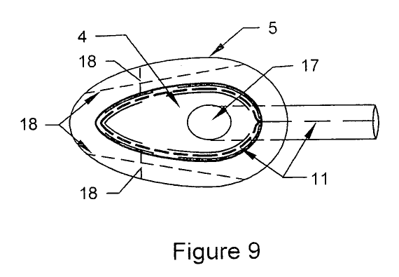

FIG. 9 shows a perspective view of strategically placed creases on the laryngeal face of the endplate.

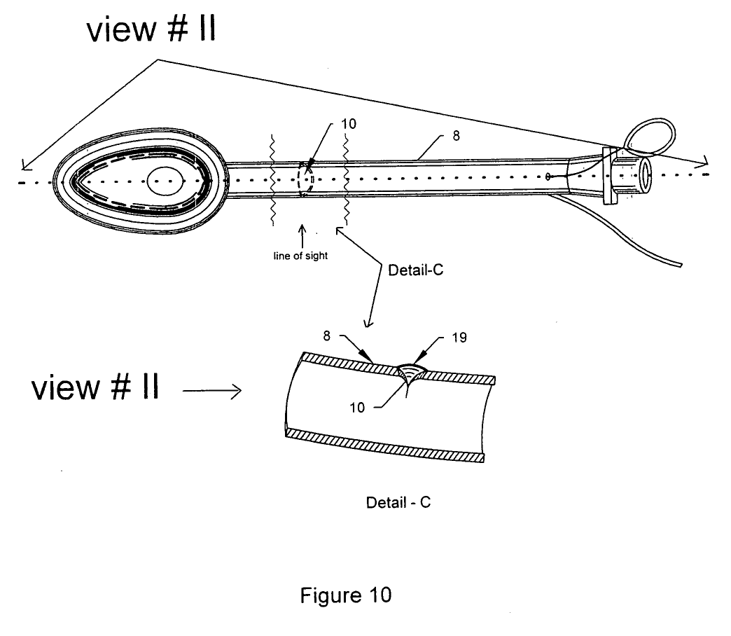

FIG. 10 shows a detail perspective anterior view of a strategically placed tubular notch on the gas tube, and detail C shows a half section of the gas tube along the dotted line that runs through the tubular notch.

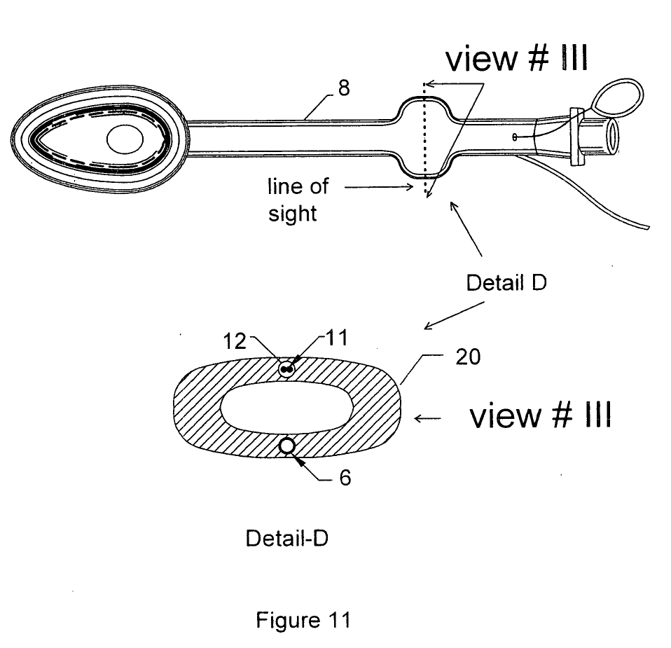

FIG. 11 shows detail of a gas tube geometric design in an anterior view of the airway device with detail D showing a cross section of the gas tube along the dotted line that runs through a point where teeth would contact the gas tube.

### DETAILED DESCRIPTION OF THE DRAWINGS

Other objects, features and advantages of the invention will become apparent from the following description of the preferred embodiments when taken in conjunction with the accompanying drawings.

FIG. 1 through 3 show three views of the airway device. The three basic components of this airway include the endplate #1, gas tube #8 and gas tube connector #9.

FIG. 1 shows the posterior view of the device that includes the pharyngeal side #3 and the outer rim cuff #5 of the endplate #1. The posterior side of the gas tube #8 and the pharyngeal side of the endplate would rest against the posterior oral pharynx when in the normal inserted position. The pilot balloon and valve #7 is used to inflate the endplate cuff. Note that the cuff tubing #6 that inflates the cuff can be attached to the base that is larger curve portion of the endplate and can either run freely from the attachment point or run juxtaposed or within the wall of the gas tube.

FIG. 2 shows a lateral view of the airway device with a curve configuration for the gas tube #8. The concave portion is aligned to the oral cavity to improve ease of insertion. This curve can be more of a “J-curve” shape. The convex side is the posterior side that rests on the posterior oral pharynx and the concave side is the anterior side that rests on the anterior oral pharynx with the laryngeal face side #4 of the endplate #1 resting over the laryngeal opening.

FIG. 3 shows the anterior view of the airway device with the gas tube opening # 17 opened into the laryngeal face side #4 of the endplate #1 that faces the person's laryngeal opening. The guide cord #11 attached to the pull-ring #13 is guided by the guide cord conduit #12 on the anterior side of gas tube #8 wall either within or juxtaposed to the wall.

FIG. 4 shows a detailed anterior view of the laryngeal face side #4 of the endplate. This view details the guide cord #11 attachment at the endplate tip #2 and a perspective route for the guide cord. The guide cord is routed along both lateral sides of the endplate but may have alternative routes such as but not limited to the middle or along one side of the endplate. When guide cord tension is applied this will effect endplate attitude and shape. Note the gas tube opening #17 may be more oval or oblong shape at the laryngeal face side of the endplate.

FIG. 5 shows a cross sectional view of the gas tube #8. The anterior sidewall of the gas tube has the guide cord #11 within the guide cord conduit #12. This conduit is not limited to within the gas tube wall. The posterior sidewall of the gas tube has the cuff tubing #6 but may not be limited to within the gas tube wall. For example, the cuff tubing may run independently of the gas tube wall.

FIG. 6 shows two detailed, A and B, views of the anterior side of the gas tube connector #9. In detail A, note the option of a guide cord flexible sheath #15 to encase the guide cord #11 as the cord exits out the guide cord conduit exit point #14. When the pull-ring #13 is manipulated this will convey guide cord movement and effect endplate attitude and shape. Also the guide cord may have enlargement(s) #16 to limit the guide cord travel as the guide cord moves in and out the exit point.

FIG. 7 shows an operational lateral view of the airway device when the pull-ring # 13 is pulled and applies guide cord #11 action and effects endplate #1 attitude and shape. Note the endplate cuff #5 is shown deflated.

FIG. 8 shows an operational anterior view of the airway device when the pull-ring #13 is pulled and applies guide cord #11 action and effects endplate attitude and shape. The guide cord movement is limited by the guide cord enlargement(s) #16. Note one enlargement is shown with movement. The limits applied to the guide cord travel avoid under or over pull on the cord and assures optimal endplate action. Also the endplate's attitude and shape are optimized by a strategically placed crease # 18.

FIG. 9 shows detail of strategically placed endplate creases #18 on the laryngeal face side #4 of the endplate. Note the creases may be either single or multiple and located at different areas on the endplate. This figure shows creases may be mirror slants in order to create a triangular or arrowhead shape or may be perpendicular to the longitudinal axis in order to encourage anterior bending of the endplate when guide cord #11 tension is applied.

FIG. 10 shows detail of a strategically placed tubular notch #10 in an anterior view of the airway device. Also, detail C shows a half section of the gas tube #8 along the dotted line that runs through the tubular notch. Note the tubular notch has a membrane # 19 to maintain gas tube integrity. The tubular notch(es) may be either single or multiple and located on the anterior side of the gas tube to encourage anterior bending of the gas tube when guide cord tension is applied. Anterior bend of the gas tube will model closely to the natural oral pharynx curvature and therefore, facilitate the device insertion.

FIG. 11 shows detail of a gas tube #8 geometric designs in an anterior view of the airway device. Also, detail D shows a cross section of the gas tube along the dotted line that runs at a point where the teeth would contact the gas tube. The cross sectional shape may be oval to minimize mouth opening. In addition the oval shape may run the total length of the gas tube. Also note reinforcement at the lateral sides of the gas tube is designed to decrease the risk of gas tube collapse from teeth biting onto the gas tube. Also the exterior gas tube wall may have a soft texture such as a soft-jell texture particularly at the point of teeth contact.

### DETAILED DESCRIPTION OF THE INVENTION

The present art is an airway device comprised of a gas tube of suitable length with a breathing circuit connector on one end and an endplate on the other. A guide cord is attached to the endplate tip and routed through the device by guide loops or conduits and exits near the gas tube connector. The guide cord then terminates to a pull-ring or other similar finger controller.

The finger controller may have means to lock onto the gas tube. This locking onto the gas tube will allow the operator to maintain a specific applied tension on the guide cord while freeing up the operator's finger during the insertion of the airway device.

There are also strategically located guide loops or conduits that attached the guide cord onto the device. Having the guide cord attached at certain locations on the endplate and gas tube with the loops or conduits will allow specific tension to be applied onto the device when the guide cord is pulled. Pulling of the guide cord will therefore cause attitude and shape change of the airway device. Hence a desirable attitude and shape of the endplate and gas tube occurs when tension is applied to the guide cord.

Most importantly is attitude and shape control of the endplate during insertion. Consequently, the operator can apply tension to the endplate tip via pull-ring manipulation that conveys guide cord tension onto the endplate. This applied tension will maintain the endplate straight and or slightly elevated and to not fold back upon it's self. Once the airway device is placed in the oral pharynx and the endplate over the laryngeal opening the operator releases the guide cord and, thus accomplishes a successful device insertion and placement.

In addition the guide cord provides the operator dynamic control of endplate attitude and shape. Consequently, the endplate is adjustable during insertion and by effecting optimal endplate position, the operator can ease the endplate through the oral pharynx. Therefore, the operator is able to maintain the endplate in a desirable position and prevent endplate malfunction.

The said guide cord can be routed and or attached in alternative ways along the endplate. For example, the guide cord can run along one side, middle or both lateral sides of the endplate.

Particularly, the endplate diameter width can be affected when the guide cord is routed on both sides of the endplate and rejoined at the gas tube. When the operator pulls on the pull-ring to convey guide cord action the endplate width will draw in and become narrower.

The decrease in endplate width will decrease the passage resistance between the relatively smaller endplate and the surrounding oral cavity soft tissue. Thus the decrease size of the endplate eases endplate insertion and guidance through the oral cavity. Less resistance permits greater ease of endplate placement over the person's laryngeal opening.

Also, the guide cord can have means to limit travel within the device. These imposed limits or markers will avoid over and under pull of the guide cord and optimize applied tension to the guide cord.

This is accomplished by having two enlargements on the guide cord. One enlargement correlates with the neutral position and a second correlates with the optimal attitude and shape position for the endplate. These two enlargements on the guide cord would be either visualized or felt as the enlargements move next to or in and out of the guide cord conduit exit point. Alternatively, these enlargements may act as stops has the guide cord travels in and out of the exit point.

In addition, as the guide cord travels in and out of the conduit a flexible sheath can encase the cord to maintain a closed environment around the cord. If the guide cord does not have a protective covering when exiting from the conduit then contaminants may enter into the device through the space between the cord and its guide conduit. Hence having a closed environment around the guide cord will allow repeated uses of the airway device without potential contamination to the device.

In addition there are strategically placed endplate creases, gas tube notches and geometric gas tube designs that contribute to an easier endplate insertion and successful device placement.

One or more strategically placed creases on the laryngeal face side of the endplate will promote endplate bend and shape when the guide cord conveys tension. Hence, optimal shape of the airway device is conveyed by applied guide cord tension onto the endplate creases. This will add to the ease of endplate insertion and airway device placement.

Also the degree of fold or bend of the endplate along the crease line directly correlates to the strength of the endplate along the same crease axes. This adds the advantage of strength to the endplate while preserving the soft material integrity.

In particular, two creases that are mirror slants along the laryngeal side of the endplate will promote endplate strength and prevent endplate malfunction when the guide cord conveys tension. These creases would be closer at the tip and farther apart at the base of the endplate. This would give a triangular or arrowhead endplate shape and increase endplate strength so not to fold back upon itself. By enhancing endplate strength with these creases this will also permit the use of softer material for the endplate. A softer material comprising the endplate gives the advantage for a better seal over the person's laryngeal opening.

Also, one or more strategically placed tubular notches on the gas tube will promote gas tube bend when the guide cord conveys tension. This will also facilitate endplate insertion and airway device placement.

The strategically places tubular notch(es) adds the advantage of bend to the gas tube while preserving the tube strength. The degree of bend of the gas tube along the tubular notch line will not decrease the lateral strength of the gas tube. Hence, the tubular notch allows anterior bend but optimizes lateral strength of the gas tube.

In addition, geometric gas tube designs will improve airway device placement. These designs consist of a particular gas tube configuration, a suitable section of more flexible gas tube and a specific gas tube diameter.

Particularly the gas tube may have a “J-curve” shape with the bottom end of the “J-curve” attached to the endplate. This configuration is more matched to the anatomical oral cavity curvature and allows for a smooth and improved insertion of the airway device. Also, a thinner wall section of gas tube will allow more flexibility at this section. Specifically, this section is between the endplate attachment point and the end of the “J-curve” tube portion distal to the endplate attachment point. Therefore, this section of gas tube will allows for greater gas tube flexibility and conforms better to variable anatomical person length. Principally, the variations in a person's thyroid-mental distance may necessitate the airway device to flex more or less at this gas tube section. Hence, the gas tube flexibility allows for a good seat or seal over the person's laryngeal opening. Last, the gas tube will have a suitable interior diameter able to allow passage of a conventional endotracheal tube. The opportunity to pass an endotracheal tube through the laryngeal airway device will allow for the endotracheal tube to be guided to within the trachea when the said laryngeal airway device is in the proper position.

Other geometric gas tube designs will improve person safety. A gas tube with a cross sectional oval shape at a suitable point where teeth contact the gas tube will minimize month opening distance compared to a cross sectional circular shape. The greater the month opening is the more potential for jaw and temporal mandibular joint stress over time. Hence the decrease in mouth opening will reduce potential jaw and temporal mandibular joint pain. In addition having the broad side of oval shape contact the person's teeth will increase the surface area contact with the teeth but decrease the total contact pressure applied to each individual tooth when biting on the gas tube. Furthermore, an oval shape resistance to collapse from teeth biting on the gas tube will avoid gas tube obstruction. Also, a gas tube having a soft or jell texture at a suitable point where teeth contact the gas tube will decrease teeth trauma.

Another geometric gas tube design will assist with the gas tube connector. A certain suitable length of gas tube that ends to the gas tube connector may be a corrugated or other flexible tubing material to allow gas tube flex near the gas tube connection. This tube flex will allow the breathing circuit that is attached to the gas tube connector to be directed in the optimal position.

In conclusion, the main goal of the present art design will allow the operator optimal insertion and placement of the airway device with reduced risk of trauma or morbidity to the person. Furthermore, this design allows for a single-handed insertion of airway device.

A person skilled in the art may vary the invention in many ways. Such variations are not to be regarded as a departure from the spirit and scope of the invention. Additional objects, features and advantages of the invention will become apparent to those skilled in the art upon consideration of the above detailed description of the preferred embodiments exemplifying the best mode of carrying out the invention as presently perceived. All such modifications are intended to be included within the scope of the following claims.

1. An airway device having a gas tube of a certain suitable length, a breathing circuit connector on one end and an endplate of certain suitable shape and thickness on the other end with the boundaries of the endplate having a pharyngeal smooth side and a laryngeal face side with this side having the opening of the gas tube to allow gases to pass through the gas tube and exit out the laryngeal face side with said endplate having an outer inflatable rim or cuff that conforms over the person's laryngeal opening with the said cuff inflated by means of tubing connected to a pilot balloon having a universal locking check valve connector and said endplate also having a cord mechanism attached to the endplate and routed through the endplate and gas tube and exits near the gas tube connector and attached to a pull-ring with the said cord mechanism contained through out the said airway device by one or more enclosed loops, conduits or other guiding hooks to route the said cord mechanism and having said cord mechanism arranged for control by having said cord mechanism attached to a pull-ring to effect said endplate from endplate foldback or malfunction while inserting said airway.

2. An airway device having a gas tube of a certain suitable length, a breathing circuit connector on one end and an endplate of certain suitable shape and thickness on the other end with the boundaries of the endplate having a pharyngeal smooth side and a laryngeal face side with this side having the opening of the gas tube to allow gases to pass through the gas tube and exit out the laryngeal face side with said endplate having an outer inflatable rim or cuff that conforms over the person's laryngeal opening with the said cuff inflated by means of tubing connected to a pilot balloon having a universal locking check valve connector and said endplate also having one or more strategically placed creases on the laryngeal face side of the endplate for increase strength and rigidity and certain shape to the endplate to resist endplate foldback.

3. An airway device having a gas tube of a certain suitable length, a breathing circuit connector on one end and an endplate of certain suitable shape and thickness on the other end with the boundaries of the endplate having a pharyngeal smooth side and a laryngeal face side with this side having the opening of the gas tube to allow gases to pass through the gas tube and exit out the laryngeal face side with said endplate having an outer inflatable rim or cuff that conforms over the person's laryngeal opening with the said cuff inflated by means of tubing connected to a pilot balloon having a universal locking check valve connector and with said device also having one or more tubular notches to promote gas tube bend.

4. The device as described in claim 1, wherein the location of the pull-ring is located near the gas tube connector and attached to the guide cord to allow action on the said guide cord for controlling the desired endplate strength and rigidity and certain shape change and having said pull-ring able to lock onto the gas tube to maintain an applied guide cord tension.

5. The device as described in claim 1, wherein said guide cord is routed juxtaposed to or within the endplate and gas tube wall or juxtaposed to some parts and within other parts of the laryngeal airway device.

6. The device as described in claim 1, wherein said guide cord is routed through dedicated enclosed loop(s), conduit(s), tubul(es) or hook(s) guiding material to route the guide cord through the airway device for controlling and shaping of the endplate and airway device during insertion.

7. The device as described in claim 1, wherein said guide cord can be routed in alternative ways through the endplate such as through one side, middle or both lateral sides of the endplate and then routed along the gas tube and exit near the gas tube connector to said pull-ring.

8. The device as described in claim 1, wherein said guide cord is routed along both lateral sides of the said endplate and rejoined at the said gas tube so when guide cord tension is conveyed by pull-ring manipulation the endplate diameter width will decrease and effect a relative narrower endplate size.

9. The device as described in claim 2, wherein a guide cord is routed along both lateral sides of the said endplate and rejoined at the said gas tube by means of guide loops or conduits and then attached to a pull-ring near the said gas tube connector so when guide cord tension is conveyed by pull-ring manipulation will effect endplate strength and rigidity and certain shape change.

10. The device as described in claim 1, wherein the said endplate has strategically placed crease(s) placed perpendicular to the longitudinal axis on the laryngeal side to encourage increase strength and rigidity and certain shape of the endplate when cord tension is conveyed by pull-ring manipulation.

11. The device as described in claim 2, where the said endplate has strategically placed crease(s) on the laryngeal opening side or other sides to maintain ideal shape of the endplate when the cuff is in the deflated position.

12. The device as described in claim 1, where the said endplate has strategically placed creases along the laryngeal face side of the endplate with the creases either parallel or having a mirror slant being closer at the tip and farther apart at the base of the endplate and giving a triangular or arrowhead shape to the endplate when cord tension is conveyed by pull-ring manipulation.

13. The device as described in claim 1, where the said gas tube has strategically placed tubular notches to promote gas tube bend when guide cord tension is conveyed by pull-ring manipulation.

14. An airway device having a gas tube of a certain suitable length, a breathing circuit connector on one end and an endplate of certain suitable shape and thickness on the other end with the boundaries of the endplate having a pharyngeal smooth side and a laryngeal face side with this side having the opening of the gas tube to allow gases to pass through the gas tube and exit out the laryngeal face side with said endplate having an outer inflatable rim or cuff that conforms over the person's laryngeal opening with the said cuff inflated by means of tubing connected to a pilot balloon having a universal locking check valve connector and said endplate also having a cord attached to the endplate and routed through the endplate and gas tube and exits near the gas tube connector and attached to a pull-ring with the said cord contained through out the said airway device by one or more enclosed loops, conduits or other guiding material to route the said cord and having means to limit the travel of said cord within the device to avoid over and under pull of the said cord by having imposed limits, stops or markers by having an enlargement of the said cord correlating with the neutral position for the endplate and a second correlating with the optimal attitude and shape position for the endplate with these said enlargements being either visualized, felt or made has stops as the said enlargements move in and out of or next to the cord conduit exit point and having said cord pulled by the pull-ring to effect endplate from endplate foldback or malfunction and maintain desired attitude and shape while inserting said airway device.

15. An airway device having a gas tube of a certain suitable length, a breathing circuit connector on one end and an endplate of certain suitable shape and thickness on the other end with the boundaries of the endplate having a pharyngeal smooth side and a laryngeal face side with this side having the opening of the gas tube to allow gases to pass through the gas tube and exit out the laryngeal face side with said endplate having an outer inflatable rim or cuff that conforms over the person's laryngeal opening with the said cuff inflated by means of tubing connected to a pilot balloon having a universal locking check valve connector and said endplate also having a cord attached to the endplate and routed through the endplate and gas tube and exits near the gas tube connector and attached to a pull-ring with the said cord contained through out the said airway device by one or more enclosed loops, conduits or other hooks guiding material to route the said cord and having an expandable corrugated sheath to encase the said cord as the said cord exits out the guide cord conduit exit point with the expandable corrugated sheath to expand and accommodate the increase length of the pulled said cord to maintain a closed environment for the said cord.

16. A laryngeal airway device as described in claim 1, wherein said guide cord is made with a radiopaque line and or said gas tube having a “x-ray” visualization material and said device components made of autoclaviable material especially if the device is intended to be reusable.

17. A laryngeal airway device as described in claim 1, wherein materials that make up the airway device may be made of latex free material, plastic, silicone, rubber, or polymer and or have mental components mixed with nonmetal components.

18. The device as described in claim 1, wherein geometric tube designs consist of said gas tube with a cross sectional oval shape at a suitable point where teeth contact the gas tube, the gas tube having soft or jell texture at the point where teeth contact the gas tube, and a gas tube oval shape resistance to collapse from teeth biting the gas tube.

19. The device as described in claim 1, wherein geometric tube designs consist of a “J” or curve shape to said gas tube configuration to matched the anatomical oral cavity curvature, a thinner gas tube wall of suitable length between the endplate and distal end of the “J-curve” portion of the gas tube to allow relatively more flexibility and bend at this length from the rest of the gas tub and a suitable interior gas tube diameter large enough to pass through a conventional endotracheal tube.

20. The device as described in claim 1, wherein a certain suitable length of said gas tube that ends to the gas tube connector may be a corrugated or other flexible tubing material to allow gas tube flex near the gas tube connector.
